(19) United States
(12) Patent Application Publication
(10) Pub. No.: US 2013/0149351 A1
(43) Pub. Date: Jun. 13, 2013

(54) POLYMER SCAFFOLDS AND THEIR USE IN THE TREATMENT OF VISION LOSS

(71) Applicants: California Institute of Technology, Pasadena, CA (US); University of Southern California, Los Angeles, CA (US)

(72) Inventors: Choon Woo Lee, Pasadena, CA (US); Robert H. Grubbs, South Pasadena, CA (US); Paresma Patel, Frederick, MD (US); Mark Humayun, Glendale, CA (US); Kenrick Kuwahara, Cypress, CA (US); Victoria A. Piunova, Altadena, CA (US); Michael Janusz Koss, Pasadena, CA (US); Yi Zhang, Cypress, CA (US)

(73) Assignees: UNIVERSITY OF SOUTHERN CALIFORNIA, Los Angeles, CA (US); CALIFORNIA INSTITUTE OF TECHNOLOGY, Pasadena, CA (US)

(21) Appl. No.: 13/708,830

(22) Filed: Dec. 7, 2012

Related U.S. Application Data
(60) Provisional application No. 61/569,073, filed on Dec. 9, 2011.

(51) Int. Cl. A61K 9/00; C12N 5/071; C08G 63/91

(52) U.S. Cl. CPC A61K 9/00; C08G 63/912; C12N 5/0621
USPC 424/400; 528/354; 525/54.1; 424/78.3; 435/377; 435/396; 428/474.4

(57) ABSTRACT

The present invention provides for scaffolds for growing RPE cells, comprising two or more biodegradable polymers. The present invention also provides for methods for creating a scaffold for growing RPE cells. Additionally, the present invention provides for RGD peptide linked polymer scaffolds for supporting the growth of RPE cells. The present invention provides methods of culturing RPE cells using the scaffolds produced herein. The present invention also provides methods of treating vision loss through the administration of RPE cell attached RGD peptide linked polymer scaffolds produced herein. The present invention further provides kits for treating vision loss.

### CROSS REFERENCE TO RELATED APPLICATIONS

This patent application claims the benefit under 35 U.S.C. §119(e) to U.S. provisional application No. 61/569,073, filed Dec. 9, 2011, entitled Chemical Modification of Biodegradable Polymer Scaffolds Allowing for Differentiation and Attachment of Retinal Pigment Epithelial Cells Derived From Stem Cells, pending, the contents of which is incorporated by reference herein in its entirety.

### TECHNICAL FIELD

The disclosed invention is related to scaffolds for growing cells. The disclosed invention is also related to growing RPE cells. The present invention also relates to methods of treating vision loss. The present invention also relates to kits for treating vision loss.

### BACKGROUND

Biodegradable polymers such as polylactic-co-glycolic acid (PLGA), random terpolymer of poly lactide-co-glycolide-co-caprolactone (PLGC), or polycaprolactone (PCL) show good biocompatibility with controlled degradability over time inside the body. Multiple biomaterials composed of these materials are approved for clinical use. For instance, PLGA has been applied in ocular drug delivery systems. Preliminary results demonstrate that hESC-RPE can be attached and cultured on a biodegradable polymer scaffold by coating human or mouse feeder layer or proteins, such as Matrigel (or vitronectin), on the polymer surface. However, these biological coating materials may provoke immunological responses leading to graft rejection, and are logistically impractical due to lot-to-lot variability and cost of manufacturing.

Thus, there is a need for polymer scaffolds that are better able to support cell culture and adhesion for the use in stem cell based treatment for blindness.

### SUMMARY OF THE INVENTION

The present invention provides for scaffolds for growing RPE cells, comprising two or more biodegradable polymers.

The present invention provides for RGD peptide linked polymer scaffolds for supporting the growth of RPE cells, comprising an RGD peptide bonded to a linker, the linker being bonded to a polymer scaffold.

The present invention provides methods for creating a scaffold for growing RPE cells, comprising covalently linking an RGD peptide to a polymer scaffold, wherein said polymer is covalently modified and crosslinked.

The present invention provides methods for culturing RPE cells, comprising growing said RPE cells on an RGD peptide linked polymer scaffold such that said cells attach to said scaffold and remain differentiated.

The present invention provides methods for treating vision loss in a subject, comprising administering an RPE cell attached RGD peptide linked polymer scaffold to said subject in need of treatment.

The present invention provides kits, comprising a support surface, one or more polymers, a linker, a crosslinking agent, RGD peptide, and RPE cells.

The general description and the following detailed description are exemplary and explanatory only and are not restrictive of the invention, as defined in the appended claims. Other aspects of the present invention will be apparent to those skilled in the art in view of the detailed description of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale.

In the drawings:

FIG. 1, comprising FIGS. 1A-B, is a schematic representation of the formation of single layered (FIG. 1A) and multilayered (FIG. 1B) RGD peptide linked polymer scaffolds.

FIG. 2, comprising FIGS. 2A-C, is a microscopy image depicting growth of hESC-RPE cells on PLGA scaffolds. FIG. 2A depicts hESC-RPE cells 24 hours after seeding on cyclic-RGD peptide coated PLGA scaffold. FIG. 2B depicts hESC-RPE cells grown on PLGA scaffolds that were not coated with cyclic-RGD peptide. FIG. 2C depicts hESC-RPE cells 1 week after seeding on a multilayer scaffold, in which the bottom layer was a PCL scaffold and the top layer a cyclic-RGD peptide linked PLGA scaffold.

FIG. 3, comprising FIGS. 3A-B, is a microscopy image depicting the immunohistochemistry of hESC-RPE cells cultured on a cRGD peptide linked PCL-PLGA bilayer after 4 weeks. FIG. 3A depicts immunostaining for ZO-1 (Gap junction marker). FIG. 3B depicts immunostaining for RPE65 (RPE-specific marker). Both images were counter stained with DAPI (nuclear marker).

FIG. 4, comprising FIGS. 4A-C, is a microscopy image depicting growth of hESC-RPE cells on PLGC terpolymer scaffolds. FIG. 4A depicts hESC-RPE cells 4 days after after seeding. FIG. 4B depicts hESC-RPE cells 30 days after seeding. FIG. 4C depicts hESC-RPE cells 104 days after seeding. The same area of the substrate was examined after 4 (A), 30 (B) and 104 (C) days. Substrates were inspected under the optical microscope in bright phase mode.

FIG. 5, comprising FIGS. 5A-C, is a microscopy image depicting the immunocytochemistry of hESC-RPE cells grown on PLGC terpolymer scaffolds 104 days after seeding. FIG. 5A depicts staining with PMEL. FIG. 5B depicts staining with Hoechst+ZO1. FIG. 5C depicts staining with RPE65.

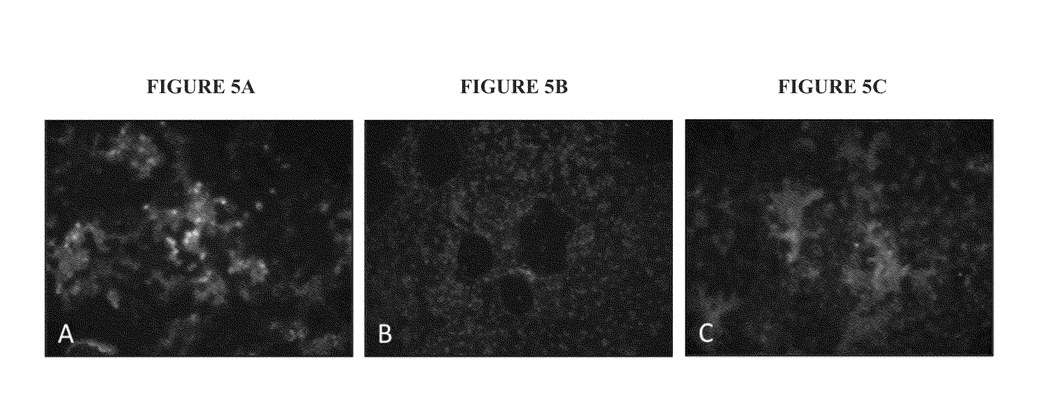

FIG. 6 is a graph depicting the permeability of polymer monolayers and bilayers.

FIG. 7 comprising FIGS. 7A-B, is a histological section of a PLGA scaffold implantation in Royal College of Surgeons (RCS) rat retinas. FIG. 7A (left) depicts staining 1 month after implantation. FIG. 7B (right) depicts staining 3 months after implantation. Images show H&E staining performed after a cryosectioning of the implanted eye. Red arrow points to PLGA film.

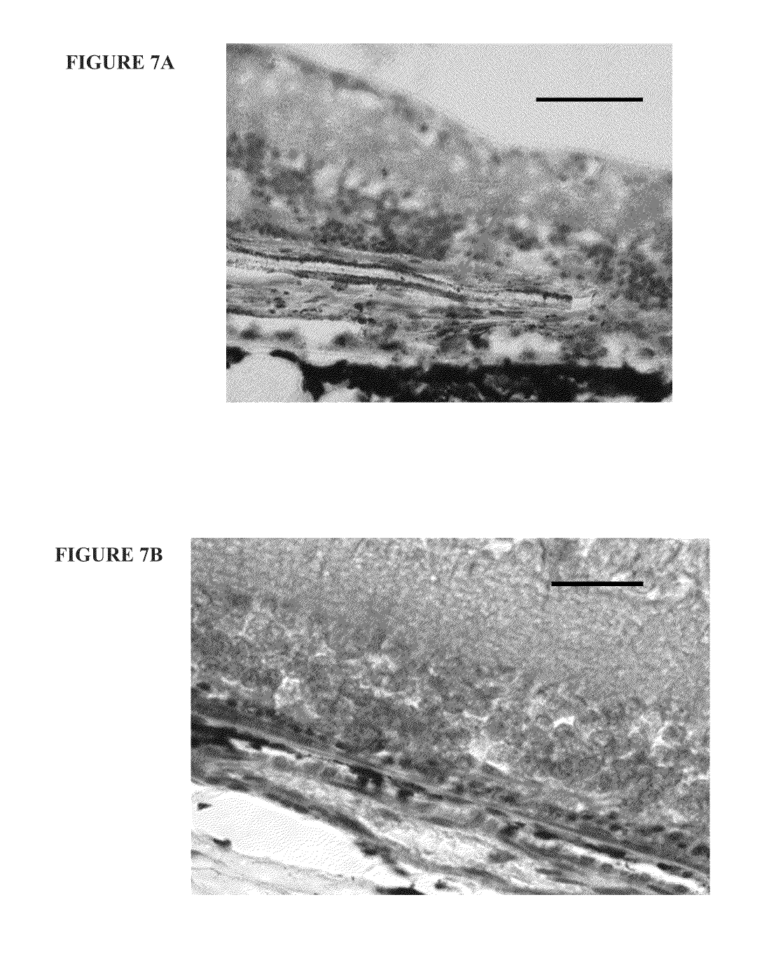

FIG. 8 is a histological slide depicting the correct placement of the polymer scaffold in the subretinal space.

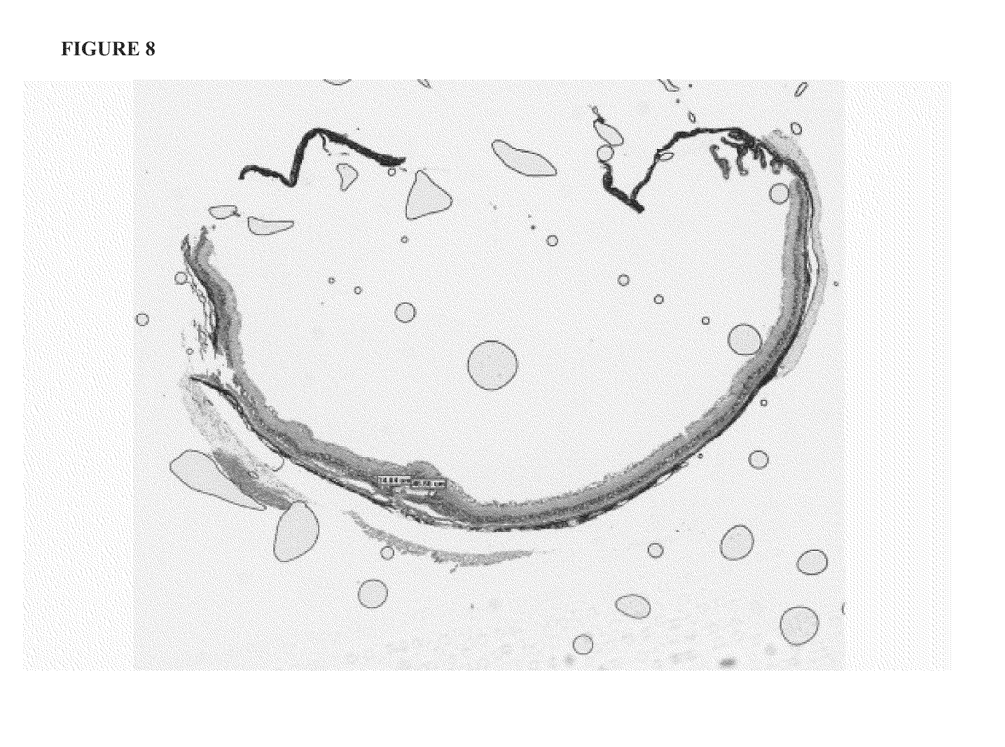

FIG. 9 depicts a PLGA/PCL bilayer (PLGA top; PCL bottom) in the intra-retinal space 1 month after implantation.

FIG. 10, comprising FIGS. 10A-B, depicts a PLGA monolayer 7 weeks after implantation (FIG. 10A) and a PLA monolayer 6 weeks after implantation (FIG. 10B).

FIG. 11 depicts an immune reaction to a PLGA/PCL bilayer 5 weeks post implantation. Macrophages (green), Mueller cells (red), and outer nuclear layer (blue).

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

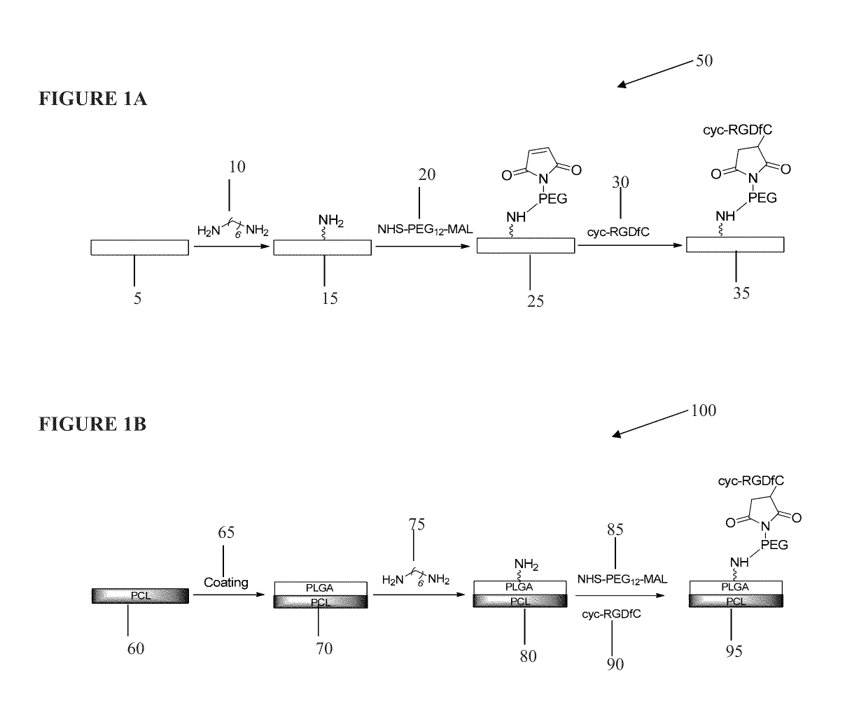

Referring now to the drawings, and FIG. 1A in particular, a single layered RGD peptide linked polymer scaffold for growing RPE cells is shown and designated as 50. The scaffold 50 has a single layer of polymer 5. The polymer layer 5 is modified by a linker 10 to form a modified polymer layer 15. The modified polymer layer 15 is crosslinked with a crosslinker 20 to form a polymer scaffold 25. The polymer scaffold 25 is incubated with an RGD peptide 30 to form a single layered RGD peptide linked polymer scaffold 35.

Referring now to FIG. 1B, a bilayered RGD peptide linked polymer scaffold for growing RPE cells is shown and designated 100. The scaffold 100 has a first layer of polymer 60. The first layer of polymer 60 is coated with a second layer of polymer 65 to form a polymer bi-layer 70. The polymer bi-layer 70 is modified by a linker 75 to form a modified polymer bi-layer 80. The modified polymer layer 80 is crosslinked with a crosslinker 85 to form a multilayered polymer scaffold. The multilayered polymer scaffold is incubated with an RGD peptide 90 to form a bilayered RGD peptide linked polymer scaffold 95.

As used herein, the term “RPE cell” refers to retinal pigment epithelial cells. It is known by those of skill in the art that RPE cells are specialized cells of the eye, located between the choroids and the neural retina. RPE cells protect, support and provide nutrition to the light sensitive photoreceptors.

RPE cells that can be grown on the scaffold of the present invention include cells that differentiate into RPE cells as well as mature RPE cells. RPE cells for use in the present invention include, but are not limited to, embryonic stem cell derived RPE cells (ESC-RPE), RPE cells derived from induced pluripotent stem cells (iPs cells or iPSCs), RPE cells derived from mesenchymal stem cells (MSC), mature RPE cells, or any combination thereof. In one example, the embryonic stem cells can be derived from a human (hESC-RPE). In another example, the embryonic stem cells can be derived from a non-human source.

As used herein, “iPSC” refers to pluripotent stem cells derived from a non-pluripotent cell source, such as an adult somatic cell. One of skill in the art would know that iPSCs can be generated by overexpressing certain genes within the cell, such as the Yamanaka factors, including Oct3/4, Sox2, Klf4, and c-Myc.

Mature RPE cells include RPE cells isolated from the retina, RPE cells grown in tissue culture, or any combination thereof.

As used herein, the term “polymer layer” refers to a layer of polymer, copolymer, blend of polymers, blend of copolymers, or any combination thereof. “Polymer layer,” “layer of polymer,” and “polymer monolayer,” are used interchangeably herein.

As used herein, the term “polymer bilayer” refers to two layers of polymer, copolymer, blend of polymers, blend of copolymers, or any combination thereof, which has a first bottom layer and a second top layer.

As used herein, the term “multilayer polymer” refers to two or more layers of polymer, copolymer, blend of polymers, blend of copolymers, of any combination thereof, which has at least a first bottom layer and a second top layer.

As used herein, the term “modified polymer layer” refers to a polymer layer that has been modified with a linker. The terms “modified polymer layer” and “linked polymer layer” are used interchangeably.

As used herein, the term “modified polymer bilayer” refers to a polymer bilayer that has been modified with a linker. The terms “modified polymer bilayer” and “linked polymer bilayer” are used interchangeably.

As used herein, the term “polymer scaffold” refers to a polymer layer that has been modified with a linker and crosslinked. Thus, “single layered polymer scaffold” refers to a single layer of polymer that has been modified with a linker and crosslinked and “multilayered polymer scaffold” refers to a two or more polymer layers that have been modified and crosslinked.

As used herein, the term “RGD peptide linked polymer scaffold” refers to a polymer monolayer that has been modified with a linker, crosslinked, and bound by an RGD peptide. Thus, “single layered RGD peptide linked polymer scaffold” refers to a single layer of polymer that has been modified with a linker, crosslinked, and bound to an RGD peptide. “Multilayered RGD peptide linked polymer scaffold” refers to two or more polymer layers that have been modified, crosslinked, and bound to an RGD peptide.

As used herein, the term “biodegradable” refers to the ability to degrade or break down inside the body. As used herein, the term “biodegradable polymer” refers to polymers that degrade or break down inside the body of a human or non-human subject.

As used herein, the term “bioabsorbable” means the ability to dissolve and be absorbed by the body. For example, a “bioabsorbable polymer” is a polymer that will dissolve and be absorbed by the body of a human or non-human subject.

The present invention relates to scaffolds for growing RPE cells, comprising two or more biodegradable polymers.

The present invention also relates to RGD peptide linked polymer scaffolds for supporting the growth of RPE cells, the scaffolds comprising an RGD peptide bonded to a linker, the linker being bonded to a polymer scaffold.

Polymers for use in the present invention include synthetic polymers, natural polymers, or both which are bioabsorbable, biodegradable, or both.

In one embodiment of the present invention, the biodegradable synthetic polymers include polyesters. Many polyesters are known in the art. Without intending to be limiting, polyesters that can be used in the present invention include forms of polylactide, polyglycolide, polycaprolactone (PCL), polyhydroxyalkanoate, polyanhydride, polyorthoester, or any combination thereof. Thus, in one embodiment, the polyester comprises polylactic-co-glycolic acid (PLGA). In another embodiment, the polyester comprises a random terpolymer of polylactide-co-glycolide-co-caprolactone (PLGC).

In another embodiment of the invention, the biodegradable polymer comprises natural polymers. Many natural polymers are known in the art. Without intending to be limiting, natural polymers that can be used in the present invention include gelatin, starch, cellulose, chitosan, hyaluronic acid, alginate, collagen, or any combination thereof.

The polymers used in the present invention can be single polymers, copolymers, or blends of different polymers, copolymers, or any combination thereof. Thus, in one embodiment of the present invention, the polymer includes a single synthetic polymer. In another embodiment, the polymer comprises a blend of synthetic polymers. For example, in a preferred aspect of the invention, the blend of synthetic polymers comprises a blend of PLGA and F127.

In one embodiment, the polymer can be a single natural polymer. In another embodiment, the polymer comprises a blend of natural polymers. In yet another embodiment, the polymer comprises a blend of synthetic and natural polymers. For example, a blend of synthetic and natural polymers for use in the present invention comprises a blend of PLGA and starch.

As used herein, the term “blend” means two or more. Thus, a blend of polymers refers to two or more polymers, copolymers or blends thereof. One with skill in the art would know that blends of polymers, copolymers or both can form different structures. For example, in one aspect of the invention, the blend can be heterogeneous. Heterogeneous refers to an unequal distribution of polymers throughout the scaffold. In another aspect of the invention, the blend can be homogenous. Homogenous refers to an equal distribution of the two or more polymers throughout the scaffold.

Using polymer blends allows one to combine different properties of different polymers. For instance, by blending different polymers, one can modify the biodegradable rate of the polymer scaffold. Also, one can use blends to create scaffolds having different permeabilities, altering the transfer of nutrients to the cell layer. Blends also allow one to control the rigidity of the scaffold, which plays a role in seeding the layer of cells upon the scaffold.

The ratio of polymer used in the blend is dependent, in part, upon the health and age of the patient and level of damage to the subretinal layer or retina itself Adjusting the ratio of polymers that comprise the scaffold can aid in the correction of subretinal environment changes due to health, age and damage.

In one aspect of the invention, the blend further comprises biocompatible polymers. Biocompatible polymers include polyethylene glycols (PEG), polypropylene glycols, and poloxamers, as well as others. Thus, in one embodiment, the polymer scaffold comprises a blend of synthetic polymers and biocompatible polymers. In another embodiment, the polymer scaffold comprises a blend of natural polymers and biocompatible polymers. In yet another embodiment, the polymer scaffold comprises a blend of synthetic polymers, natural polymers, and biocompatible polymers.

Suitable ratios of synthetic or natural polymer to biocompatible polymer used in the scaffold according to the present invention includes ratios at or below 9:1, and more preferably at or below 4:1. Thus, in one aspect of the invention, the amount of PEG used comprises 20 percent of the total weight of the blend (20 weight %). In other embodiments, the amount of PEG can be less than 20 weight percent.

The polymer scaffolds used in the present invention includes scaffolds that are single layered. Thus, in one embodiment, the scaffold comprises a single layered polymer. In another embodiment, the scaffold comprises a single layered copolymer. In yet another embodiment, the scaffold can be a single layer comprising a blend of polymers and/or copolymers.

The polymer scaffolds of the present invention also includes scaffolds that contain multiple layers. Thus, for example, in one aspect of the invention the multiple layer scaffold comprises two layers. In another aspect of the invention, the multiple layered scaffold comprises three layers. And so on. In one embodiment, the multiple layered scaffolds includes scaffolds in which each layer can be composed of the same polymer, copolymer, or blend of polymers and/or copolymers. In another embodiment, the multiple layered scaffold includes scaffolds in which each layer can be composed of different polymers, copolymers, or blends of polymers and/or copolymers.

The use of scaffolds with multiple layers of different polymers, copolymers or blends provides variability in the biodegradable rate of the scaffold. For example, multiple layered scaffolds can be generated such that the rate of degradation of the first layer is faster or slower than the rate of degradation of the second layer. In one embodiment of the present invention, the multiple layer scaffold comprises a first layer of polycaprolactone and second layer of polylactic-co-glycolic acid.

Multiple layer scaffolds according to the present invention include scaffolds in which each layer comprises an equal ratio of polymer. Alternatively, the multiple layer scaffolds described herein include scaffolds in which each layer comprises an unequal ratio of polymer.

Polymer scaffolds according to the present invention can be created at various thicknesses. The thickness of the polymer scaffold will also influence the rate of degradation. Suitable thicknesses include polymer scaffolds within the range of about 1 μm to about 50 μm, preferably within the range of about 3 μm to about 25 μm, and even more preferably within the range of 5 μm to about 10 μm.

The present invention also provides methods of creating a scaffold for growing RPE cells, the method comprising linking an RGD peptide to a polymer scaffold, wherein the polymer is modified and crosslinked.

In one aspect of the invention, the polymer layers can be formed upon a support surface. Formation of a layer of polymer upon a support surface can be performed by various techniques well known to those with skill in the art. In one aspect of the method disclosed herein, the polymer layer can be formed by casting. In one example of the casting method, a solution of polymer can be added onto a support surface, allowed to air dry, and then dried under a vacuum. The thickness of the resulting polymer layer depends upon several factors, including the concentration of polymer, the amount of polymer solution added, and the ratio of polymers if blended.

The polymer layer can be formed by any number of thin film coating methods such as spin coating. In one example of the spin coating method, a solution of polymer can be added onto a support surface and spun in a spin coating machine. The speed of the spin coating machine can vary depending upon the desired thickness of the polymer scaffold. For example, the spinning step can be performed between 1000 and 3000 rpm. The coated support surface can be dried under vacuum overnight. The thickness of the resulting polymer layer depends upon several factors, including speed of the spinning step and concentration of the polymer solution.

The polymer layer can be formed using a solid-liquid phase technique. In one example of the solid-liquid phase technique, a polymer bilayer can be formed on dioxan. The polymers can be spread uniformly on a glass slide and placed on an ice bath. After 1 min a copper wire, which had been sitting in dry ice, touches the surface of the glass slide and initiates nucleation of dioxane crystals. Once the dioxane is solidified the slide can be placed in a freezer at −20° C. for one hour. To sublimate the dioxane the slides can be placed on a lyophilizer.

The polymer layer can be formed by a phase-inversion technique. In one example of the phase-inversion technique, two polymer solutions can be mixed with glycerol to promote large pore formation and placed on a glass slide. The polymer coated slides can be then immersed with 18 Mega Ohm water at room temperature for 10 minutes.

Multiple layered scaffolds can be created using any of the above procedures. In one embodiment, all layers can be formed using the casting method. In another embodiment, all layers can be formed using spin coating. In another embodiment, the first layer of the multiple layer scaffold can be created by casting and the second layer can be created by spin coating. In yet another embodiment, the first layer can be created by spin coating and the second layer can be created by casting. To make multi-layer scaffolds, the casting or spin coating procedure can be performed and repeated once the previous layer is completely dry. Multiple layered scaffolds can also be formed using either the solid-liquid phase separation technique, phase-inversion technique, or both.

Scaffold thicknesses can be measured by various methods known in the art, such as through the use of a stylus profilometer.

As used herein, “polymer solution” refers to one or more polymers of the present invention dissolved in a liquid. One of skill in the art would know that multiple types of solvents can be used to dissolve polymers. For example, in one embodiment of the claimed method, the polymer can be dissolved in a suitable solvent such as tetrachloroethane prior to forming the polymer layer. In another embodiment of the claimed method, the polymer can be dissolved in chloroform prior to said coating step.

As used herein, “support surface” refers to a surface used in the formation of a layer of polymer. Support surfaces used in the claimed invention include any rigid and uniform surface, including, but not limited to, glass, plastic, metal, and silicon.

As used herein, “cover glass” refers to a glass support surface used in the formation of the polymer scaffold.

The formation of the polymer layers can be performed at a variety of temperatures dependent upon the melting temperature of the polymer. Suitable temperatures can be between about 5° C. to about 50° C., more preferably between about 15° C. to about 35° C., and more preferably between about 20° C. to about 25° C. It is known by those of skill in the art that 20° C. to 25° C. is room temperature.

Various polymer concentrations can be used to create the polymer scaffold. In one aspect of the invention, the polymer concentration ranges from about 0.05 g/ml to about 0.5 g/ml.

Covalent modification of the polymer layer can be performed by various methods known to those of skill in the art. In one embodiment, the polymer can be incubated with a suitable linker. Multiple linkers can be used in the present invention. Suitable linkers include di-, tri-, or multi-functional linkers. Suitable di-funtional linkers include alkyldiamines having from 2 to 2000 carbon atoms, preferably from 4 to 100 and more preferably from 6 to 10 carbon atoms. In a particularly preferred embodiment a suitable alkyldiamine linker comprises 1,6-hexamethylenediamine. Other suitable linkers include, but are not limited to, diamino alkane, diamino alkene, aminothiols, or any combination thereof.

Incubation of the polymer layer with the linker can be performed at various temperatures. Suitable temperatures can be between about 5° C. to about 50° C., more preferably between about 15° C. to about 35° C., and more preferably between about 20° C. to about 25° C. Thus, in one embodiment, the polymer can be incubated with the linkers at room temperature.

In one aspect of the invention, the modified polymer layer can be crosslinked. Crosslinking can be performed using a variety of techniques know in the art. In one aspect of the claimed method, the crosslinking step can be performed using a solution comprising NHS-PEG₁₂-Maleimide.

Crosslinking can be performed at a variety of temperatures. Suitable temperatures can be between about 5° C. to about 50° C., more preferably between about 15° C. to about 35° C., and more preferably between about 20° C. to about 25° C. It is known by those of skill in the art that 20° C. to 25° C. is room temperature.

Crosslinking of the modified polymer layer can be performed at a variety of pH. In one application of the claimed method, the crosslinking can be carried out at or around pH 7.0. In a preferred application of the claimed method, crosslinking can be carried out above pH 7.0.

In a preferred aspect of the invention, the scaffold contains RGD peptides. As used herein, the term “RGD peptide” refers to proteins or peptides containing an arginine-glycine-aspartic acid sequence. In the art, such peptides are also referred to as arginylglycylaspartic acid. The RGD peptide used in the present invention includes small peptides composed primarily of an RGD sequence, long peptides or full length proteins which contain an RGD peptide sequence, or both. Thus, in one embodiment of the current invention, the RGD peptide can be a cyclic-RGD peptide (cRGD). In another embodiment of the current invention, the RGD peptide can be a non-cyclic RGD peptide. In yet another embodiment of the present invention, the RGD peptide can be a protein or peptide containing an RGD sequence. For example, the protein containing the RGD sequence can be an extracellular matrix protein involved in extracellular recognition. In one aspect of the invention, the protein containing an RGD sequence can be vitronectin.

RGD peptides can be bound to the scaffold using a variety of techniques known in the art. In a preferred aspect of the invention, the RGD peptide can be covalently bound to crosslinked, diamine modified polymer. This can be achieved by incubating a solution of RGD peptide with the polymer scaffold. The incubation can be performed at a variety of conditions, including temperature, pH, and concentration. Suitable temperatures can be between about 5° C. to about 50° C., more preferably between about 15° C. to about 35° C., and more preferably between about 20° C. to about 25° C. The pH of the RGD peptide used in the incubation step can vary. Suitable pHs of the peptide can be in the range of from about 3 to 11, preferably in the range of from about 6 to 8 pH, and preferably at or around pH 7.0. In one preferred aspect of the invention, the pH of peptide can be 7.2.

The concentration of the RGD peptide can vary. It is preferred that the concentration of the RGD peptide is such that the peptide saturates the binding sites of NHS-PEG maleimide. Suitable concentrations can be in the range of about 0.001 mg/ml to about 5.0 mg/ml, preferably about 0.01 mg/ml to about 1.0 mg/ml, and more preferably about 0.1 mg/ml to about 0.5 mg/ml. In a preferred aspect of the claimed method, the concentration of the RGD peptide can be at or around 0.1 mg/ml.

RPE cells interact with and attach to the polymer scaffold. In a preferred aspect of the invention, the RGD peptide promotes the adhesion of RPE cells to the scaffold and the subsequent differentiation of the RPE cells. RPE cells can bind to the RGD peptide through a variety of different interactions. For example, RPE cells can interact with the RGD peptides through proteins expressed on the surface of the cells. In one aspect of the invention, the RPE cells can interact with the RGD peptide through integrin-peptide ligand interactions. It would be known by one with skill in the art that numerous bonds are involved in protein-protein interactions. These bonds include, but are not limited to, covalent bonds, hydrogen bonds, electrostatic interactions, hydrophobic and hydrophilic interactions, Van der Waals forces, and any combination thereof.

The polymer layers can be removed from the support surface prior to the subsequent modification steps. For example, the polymer layers can be removed from the support surface prior to the covalent modification of the polymer. In one embodiment, the polymer layers and support surface are soaked in water overnight, resulting in the detachment of the polymer layers. Alternatively, the polymer layers can be removed from the support surface during the covalent modification step. Thus, in one embodiment, the polymer scaffold can be detached from the support surface while being incubated in the 1,6-hexamethylenediamine solution.

The scaffold can be removed from the support surface after formation of the RGD peptide linked polymer scaffold. Thus, in one embodiment, the scaffold is not removed from the support surface until after the RGD peptide is bound to the crosslinked, modified polymer scaffold.

The present invention also provides methods of culturing RPE cells, comprising growing the RPE cells on RGD peptide linked polymer scaffolds such that the cells attach to the scaffold and remain differentiated.

In one aspect of the claimed method, the RPE cells continue to proliferate and form a monolayer after attaching to the polymer scaffold. The polymer scaffold allows one to create RPE cell monolayers of various confluences. For example, the RGD peptide linked polymer scaffold of the present method can be used to create a monolayer of RPE cells that is at least 25% confluent, preferably at least 50% confluent, and more preferably at least 90% to 95% confluent.

The method of the present invention allows one to maintain differentiated RPE cells for extended periods of time. In one embodiment, the RPE cells remain differentiated for 4 days. In another embodiment, the RPE cells remain differentiated for 30 days. In another embodiment, the RPE cells remain differentiated for 104 days.

Various RPE cells can be cultured according to the claimed method. These include, but are not limited to, hESC-RPE, non-human ESC-RPE, iPSC derived RPE cells, adult stem cell derived RPE cells, and mature RPE cells.

RPE cells for use in the claimed culturing method include cells that are differentiated before the cells attach to the polymer scaffold. For example, in one embodiment of the claimed method, hESC-RPE cells can be added to the polymer scaffold and cultured. RPE cells for use in the claimed method also include cells that are not differentiated prior to attaching, but instead differentiate after attaching to the polymer scaffold. For example, in one embodiment of the claimed method, hESCs can be added to the polymer scaffold, attach, and differentiate into hESC-RPE cells.

The method of the claimed invention can be used to grow RPE cells that were previously being cultured. For example, cells cultured in a petri dish, flask or other culturing device are trypsinized and transferred to the polymer scaffold. Thus, in one embodiment, cells can be trypsinized prior to adding the cells to the polymer scaffold.

In order to form a confluent monolayer of cells, it is preferred to add cells to the scaffold at a concentration of at least 100,000 cells/ml, more preferably at a concentration of at least 300,000 cells/ml, and even more preferably at a concentration of at least 500,000 cells/ml.

The method of the claimed invention can be used for a variety of purposes. In one embodiment, the cultured cells can be used to study biochemical properties of RPE cells. In another embodiment, the cultured RPE cells can be used in cell based treatment of vision loss.

One with skill in the art would know the proper conditions to culture cells. In one aspect of the invention, cells can be cultured at 37° C., pH 7.2, 5% CO₂, in XVIVO10 media.

The present invention also provides methods of treating vision loss in a subject, comprising administering a RPE cell attached RGD peptide linked polymer scaffold to a subject in need of treatment.

As used herein, the term “subject” is intended to mean any mammal Thus the method of the present invention is applicable to human and nonhuman subjects, although it is most preferably used in humans. Thus, in one embodiment, the subject treated using the method of the present invention can be a human. In another embodiment, the subject treated using the method of the present invention can be a rat. In yet another embodiment, the subject treated can be another mammal “Subject” and “patient” are used interchangeably herein.

As used herein, the terms “treating” and “treatment” include and encompass reducing, ameliorating, alleviating, reversing, inhibiting, preventing and/or eliminating vision loss and promoting, inducing, stimulating and/or supporting sight. “Treating” and “treatment” also refer to reduction in severity and/or frequency of symptoms, elimination of symptoms and/or underlying cause, prevention of the occurrence of symptoms and/or their underlying cause, and improvement or remediation of damage.

In one embodiment of the claimed method the RGD peptide linked polymer scaffold comprises a RGD peptide covalently linked to a crosslinked, 1,6-hexamethylenediamine modified polymer.

The RPE cells attached to the RGD peptide linked polymer scaffold can be obtained from a variety of sources. In one embodiment, the RPE cells can be derived from the subject in need of treatment. In one example, mature RPE cells can be obtained from the subject, cultured on RGD peptide linked polymer scaffolds, and administered back into the subject. In another example, adult stem cells can be isolated from the subject in need of treatment, differentiated into RPE cells, cultured on RGD peptide linked polymer scaffolds, and administered back into the subject. In another example, fibroblast cells can be isolated from the subject, induced to form RPE cells (such as iPSC derived RPE cells), cultured on RGD peptide linked polymer scaffolds, and administered back into the subject.

In another embodiment, the RPE cells can be derived from a source other than the subject in need of treatment. For example, embryonic stem cells can be obtained from existing cell lines, such as the H1 or H9 cell lines, cultured on RGD peptide linked polymer scaffolds, and administered to the subject in need of treatment. In another example, new embryonic stem cell lines can be created, cultured on RGD peptide linked polymer scaffolds, and administered to the subject in need of treatment. In another example, RPE cells or cells that can be differentiated into RPE cells can be obtained from a subject other than the subject in need of treatment, cultured on RGD peptide linked polymer scaffolds, and administered to the subject in need of treatment.

As used herein, the term “vision loss” refers to reduction in sight and includes partial and complete loss or reduction in sight.

The method of the present invention can be used to treat vision loss resulting from numerous causes. In one application of the method, the vision loss treated can be caused by macular degeneration. In another application, the vision loss treated can be caused by retinopathy. In yet another aspect, the vision loss treated can be caused by Stargardt's macular dystrophy. In another aspect of the invention, the vision loss treated can be caused by retinal detachment.

As used herein, the term “retinopathy” refers to damage to the retina and includes acute retinopathy and persistent retinopathy. One of skill in the art would appreciate that retinopathy has numerous causes, including but not limited to, circinate retinopathy, diabetic retinopathy, renal retinopathy, hypertensive retinopathy, exudative retinopathy, retinopathy of prematurity, radiation retinopathy, sickle-cell retinopathy, and stellate retinopathy.

In one aspect of the claimed method, the RPE cell attached RGD linked polymer scaffold can be administered to the subject's eye by subretinal implantation. For example, RPE cells can be cultured on the RGD peptide linked polymer scaffold until the cells form a confluent monolayer. In one embodiment, the cells can be 100% confluent prior to administration. In another embodiment, the cells can be 90-95% prior to implantation. In another embodiment, the cells can be macroscopically confluent prior to implantation. The term “microscopically confluent” or “microscopic confluency” as used herein, refers to cell attached scaffolds in which small portions have less confluency or a lower differentiation compared to other parts.

The RPE cell attached RGD peptide linked polymer scaffold of the claimed method can be cultured for varying amounts of time prior to implantation. It is preferred that the cells remain differentiated prior to implantation. The growth time should be such that the cells are not too old prior to implantation, but are grown long enough to express the proper phenotypic markers and form a developed epithelial sheet. Cells can be grown on the scaffold for 1 to 4 weeks prior to implantation, preferably 2 to 8 weeks prior to implantation, and more preferably 4 to 6 weeks prior to implantation.

Subretinal implantation can be performed by a variety of techniques known in the art. In one example, the subretinal implantation can be performed after fixation of the superior and temporal rectus muscles, sclerotomy, and detachment of the retina.

The polymer scaffolds of the claimed invention have an elastic modulus that is strong enough to withstand the chemical and biological modifications and the subretinal implantation procedure. Those with skill in the art know that the term “elastic modulus” refers to the ratio of force exerted upon an object to that object's resulting deformation, and is a measure of an objects tendency to deform when a force is applied to it. Elastic modulus and modulus of elasticity are used interchangeably herein. One measure an object's elastic modulus is the Young's modulus. Those with skill in the art would appreciate that the term “Young's modulus” refers to the ratio of tensile strength to tensile strain, and is therefore a constant of proportionality associated with the change in length of a material according to its elastic properties. In one aspect of the present invention, the polymer scaffold has an elastic modulus that mimics the elastic modulus of the subject's retina. In a preferred aspect of the invention, the elastic modulus of the polymer scaffold can be equal to or around 0.1 mPa. In a more preferred aspect of the invention, the Young's modulus can be equal to or around 0.1 mPa.

In another aspect of the claimed invention, the polymer scaffold possesses a maximum strain less than a maximum strain of said subject's retina. It is preferred that the maximum strain of the scaffold is lower than the eye prior to implantation and more preferred that the maximum strain of the scaffold is lower than the eye after implantation. It is preferred that the maximum strain of the implanted scaffold is 83% of the strain of the eye, and even more preferred that the maximum strain of the implanted scaffold is less than 83% of the strain of the eye.

The RPE cell attached RGD peptide linked polymer scaffold can be various sizes depending on the subject's needs. In one embodiment, the size of the scaffold can be adjusted prior to implantation.

It is preferred that the RPE cell attached polymer scaffolds of the present invention maintain permeability after subretinal implantation to support the growth of the RPE cells. Thus, in one application of the claimed method, the RPE cell attached RGD polymer scaffold remains permeable to nutrients and oxygen after implantation.

Following subretinal implantation, the scaffolds of the present invention degrade. The rate of degradation of the scaffold is dependent upon the scaffold's composition. Factors that will influence the rate of degradation include, but are not limited to, the number of layers, the polymer or blend of polymers, and the thickness of the scaffold. In one aspect of the invention, the scaffold degrades within one month following implantation. In a preferred aspect of the invention, the scaffold degrades after one month following implantation. In a more preferred aspect of the invention, the scaffold degrades 4 to 6 months after implantation.

In a preferred aspect of the invention, the RPE cells can be incorporated into the eye of the subject in need of treatment following degradation and support photoreceptor survival.

The polymer scaffold can be coated with an agent to prevent an adverse reaction in the subject undergoing treatment. As used herein, the term “coated” or “coating” or “coat” means apply to. The coating can be transient, such as dipping the scaffold into the agent, or it can be long lasting, such as chemically binding the agent to the scaffold. As used herein, the term “adverse reaction” refers to any unwanted side effects caused by, or potentially caused by, the implantation procedure. Thus, the agent can be used to prevent viral, bacterial, fungal infections, graft-versus-host disease, as well as others.

One with skill in the art would know that multiple types of substances can be used to help prevent an adverse reaction in a subject during a surgical procedure. Examples include, but are not limited to, immunosuppressants, anti-inflammatory compounds, antibacterial compounds, anti-viral compounds, anti-fungal compounds, or any combination thereof. Thus, in one aspect of the present invention, the polymer scaffold can coated with an antibacterial compound to prevent an adverse reaction in the subject being treated. In another aspect, the polymer scaffold can be coated with an immunosuppressant to prevent an adverse reaction in the subject being treated.

The polymer scaffolds of the present invention can be coated with the agent at various times. In one aspect of the invention, the scaffold can be coated with the agent prior to administration of the polymer scaffold. For example, the polymer scaffold can be coated during the scaffold's formation. In another example, the polymer scaffold can be coated after formation but prior to implantation. In another aspect of the invention, the scaffold can be coated contemporaneously with administration of the polymer scaffold. For example, the surgeon can apply the agent to the scaffold while performing the operation.

Agents to prevent adverse reactions in the subject can also be placed within the polymer scaffold. For example, spheres or traces of suitable agent can be placed within the polymer scaffold during formation. In one aspect of the invention, the agent is placed within the polymer layer during formation of the polymer layers. Placing agent within the scaffold allows the agent to be released locally in the subject upon degradation of the RGD peptide linked polymer scaffold. Placing agent within the scaffold also allows one to control the rate of release of the agent. For example, upon degradation of the scaffold, the release of the agent can be a delayed release, extended release, rapid release, or any combination thereof.

The present invention additionally provides a kit comprising a support surface, one or more polymers, a linker, a crosslinking agent, RGD peptide, and RPE cells.

The kit of the present invention includes polymers to be used for the formation of a polymer scaffold. Any of the polymers or combinations thereof disclosed herein are appropriate to use in the kit of the present invention. In one aspect of the invention, the polymers are capable of being dissolved in a solution. For example, the kit of the present invention comprises polymers in solid form capable of being dissolved in tetrachloroethane, chloroform, or both.

The kit also contains suitable crosslinking agents and linkers. The crosslinking agents and linkers disclosed herein are appropriate to use in the kit of the present invention

The kit also contains RGD peptide to be bound to the polymer surface. In one aspect of the present invention, the RGD peptide can be in solid form. In another aspect, the RGD peptide can be in solution.

In one embodiment, the kit further contains additional solutions to assist in carrying out the formation of the polymer scaffold, including phosphate buffered saline, isopropanol, ethanol or any combination thereof.

In one aspect of the kit, the RPE cells can be in a frozen state. The kit further contains a coolant to maintain the cells in a frozen/cryopreserved state. One with skill in the art would understand how to maintain cells in a cryopreserved state. Coolants for use in the present kit include, but are not limited to, ice packs, dry ice, liquid nitrogen or any combination thereof.

When ranges are used herein for physical properties, such as molecular weight, or chemical properties, such as chemical formulae, all combinations, and subcombinations of ranges for specific embodiments therein are intended to be included.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### EXAMPLES

### Example 1

### Formation of Cyclic-RGD Linked Peptide Coated Polymer Scaffold

A chloroform solution of PLGA copolymer (85:15) or PLGC (70:10:20) copolymer at a concentration of 25 mg/ml was casted onto a glass cover (70 μL onto 12 mm glass cover). The film was allowed to air dry, followed by drying completely under high vacuum condition. Typical thickness of film was around 5-10 μm, measured by profilometer. The polymer coated glass covers were placed in a tissue culture plate. To covalently modify the surface, a 1,6-hexamethylenediamine solution was added into the plate (1 mL solution from 1.0 mg/mL in isopropanol). The plate was shaken for 2 hour at room temperature, soaked in de-ionized water for 1 hour, and thoroughly washed with deionized water. The surface became colored during this step (blue or rainbow). The amine modified film was reacted with a cross linking agent for 5 hour while shaking at room temperature (crosslinking agent: NHS-PEG₁₂-Maleimide from Thermo Scientific, 1 mL from 1.0 mg/mL in 0.5 mM PBS buffer pH 7.2). The film was washed with de-ionized water and a solution of cyclic-RGD peptide was added and shaken overnight at room temperature (cyclo Arg-Gly-Asp-D-Phe-Cys from Peptide International, 0.1 mg in 0.5 mM PBS buffer pH 7.2). The resulting cyclic-RGD linked polymer scaffold was washed with de-ionized water.

### Example 2

### Use of Cyclic-RGD Peptide Linked Polymer Scaffolds for Culturing hESC-RPE Cells

Cyclic-RGD peptide linked polymer scaffolds were cut into 6 mm diameter discs and placed into wells of a 96 well-plate. The discs were incubated at room temperature for 1 hour with 70% ethanol solution to prevent contamination. The discs were washed for two 15 min washes with PBS. hESC-RPE cells were trypsinized, washed twice with 10 mL of PBS, and adjusted to a concentration of 500,000 cell/mL. 100 μL of hESC-RPE was deposited to each disc containing well. The cells were incubated overnight at 37° C. at 5% CO₂. The resulting cell attachment were observed and recorded.

### Results

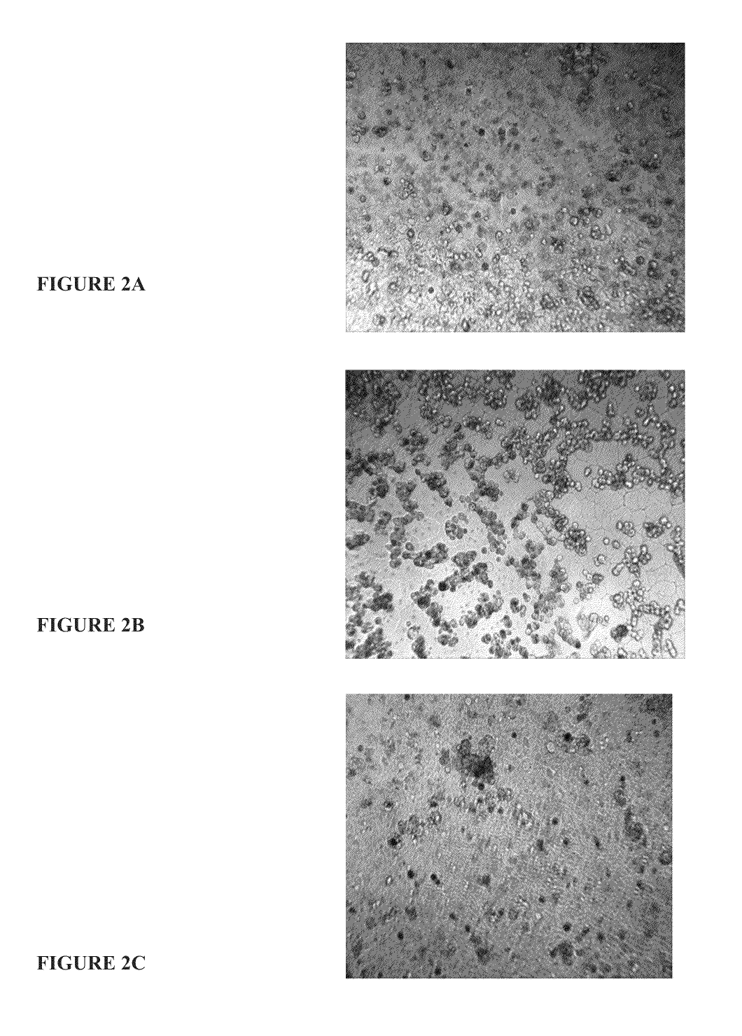

After 24 hours from the seeding time, hESC-RPE cells appeared to be attached to the modified PLGA scaffold with 80 to 90% confluence. The cells exhibited a flat cuboidal (cobblestone) morphology confirming cell attachment (FIG. 2A). Scaffolds without cyclic RGD were unable to retain cell attachment as shown by the spherical cell morphology on these substrates. (FIG. 2B). After one week, hESC-RPE cells on a multilayered scaffold comprising a bottom PCL layer and a top cyclic-RGD peptide coated PLGA layer (FIG. 2C) remained well attached without dedifferentiation into fibroblast like cells.

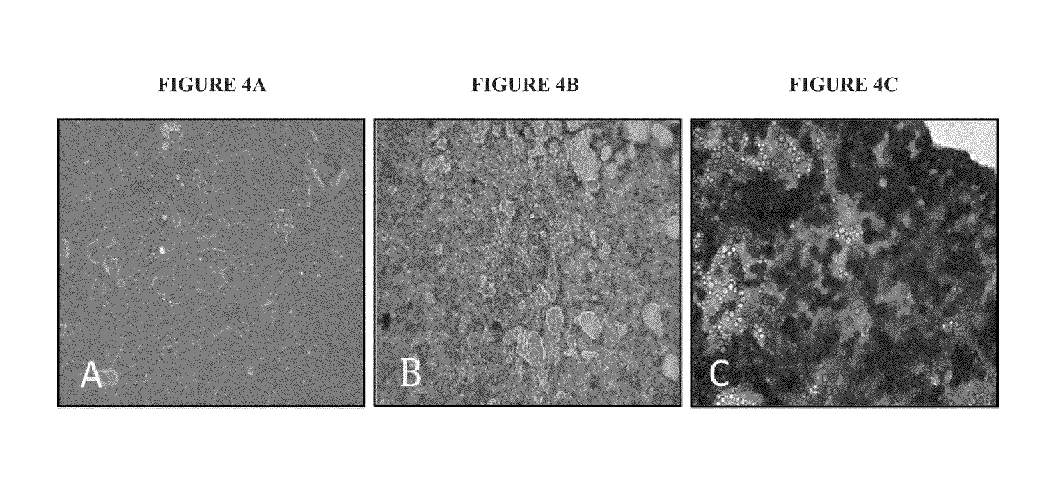

After 4 days from seeding time, the optical microscope images showed abundant cell attachment and formation of an almost confluent monolayer of cells with characteristic cobblestone morphology (FIG. 4A). At day 30, the majority of hESC-RPE cells remained attached, however, the formation of a few small patches, where cells detached, were noted (FIG. 4B). At day 104 after seeding, the cells showed formation of almost (˜90-95%) confluent monolayer of well-pigmented cells with characteristic cobblestone morphology (FIG. 4C).

### Example 3

### Immunocytochemistry of Polymer Scaffolds

### PCL-PLGA-cRGD Linked Scaffolds

To observe the RPE phenotypes of the hESC-RPE cells seeded on a thin film of PCL-PLGA-cRGD peptide linked scaffold, ICC stainings were performed. The cell attached PCL-PLGA-cRGD scaffolds were washed twice with PBS and then fixed with 2% paraformaldehyde in PBS for 20 mins at RT. The fixed scaffold was rinsed twice with 0.1% BSA in PBS (wash buffer) and incubated with a blocking agent of 10% goat serum 0.3% triton-X 100 for 40 mins. The scaffolds were incubated with either ZO-1 (1:200 dilution, Invitrogen) or RPE-65 (1:500 dilution, Millipore) overnight at 4° C. After three 10 min washes at RT with wash buffer, the scaffold was incubated with Cy5-conjugated anti-rabbit IgG secondary antibody for 30 mins. Three more 10min washes with the wash buffer were performed followed by 3 μM DAPI solution for the counterstain. The scaffold was viewed under a fluorescent microscope.

### PLGC-cRGD Peptide Linked Scaffolds

Scaffolds were cut into approximately 0.5 cm² pieces and each piece was placed into a disposable beaker, covered with 1 ml of 1× PBS, and placed on rocker in 4° C. for 5 minutes. Washing was repeated twice each time with fresh 1× PBS followed by blocking with freshly prepared block-solution (1% BSA, 1% Goat Serum, 0.1% NP-40 in 1× PBS) at 4° C. on rocker. After 1 hour the block was removed, primary antibodies were added and the samples were incubated on rocker in 4° C. overnight. The samples were washed with 1× PBS 3 times, 5 minutes each in 4° C. on rocker. Buffer was replaced with secondary antibodies and samples were incubated again on rocker for 1 hour at room temperature. The samples were washed 3 times with 1× PBS, secondary antibodies were replaced with Hoechst (final 8 ug/ml in 1× PBS) and the samples were incubated for 5 minutes at room temperature on rocker. After the samples were mounted in Prolong Anti-Fade mounting medium, they were cured for 24 hours at room temperature in the dark and imaged on Olympus BX51 with QCapture Pro software.

### Results

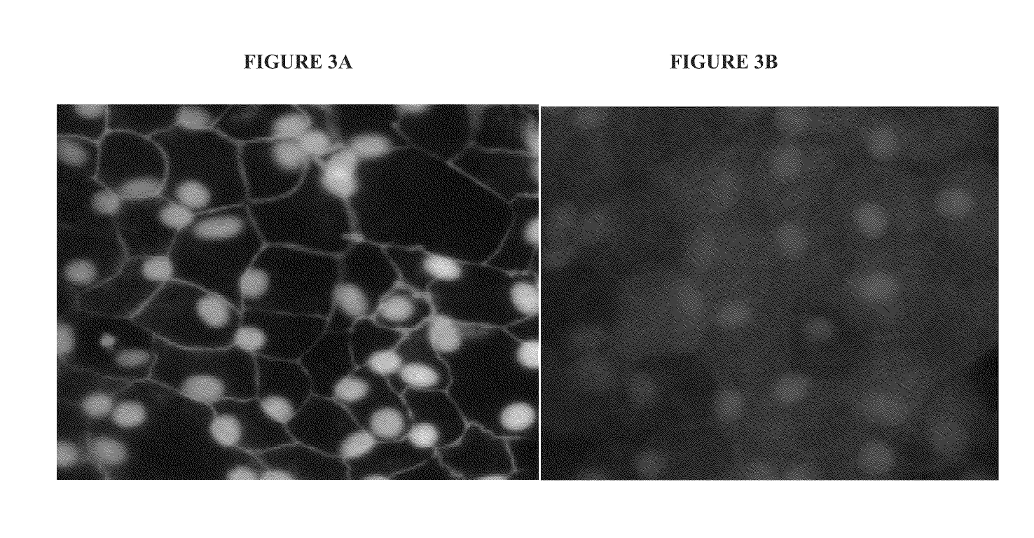

Immunocytochemistry shows that the hESC-RPE cells, after 4 weeks of culturing on PCL-PLGA-cRGD, still maintain their morphology and phenotype of a retinal epithelium (FIG. 3).

### Example 4

### Preparation of Scaffolds by Spin Coating

Polymers were dissolved Tetrachloroethane (TCE) or Chloroform (0.05˜0.5 g/mL), and loaded (0.1˜0.5 mL) onto cover glass (12 mm or 24 mm) The polymer loaded cover glass was placed onto the spin coating machine. The speed of coating was adjusted around between 1000 and 3000 rpm. The thickness depends on the speed and concentration. The coated cover glass was dried under vacuum for overnight. For making multi-layer scaffolds, the spin coating procedure was repeated on the dried single layer of polymer. After completely drying, thickness of polymer layers was measured by using a stylus profilometer.

### Example 5

### Preparation of Polymer Layers and Bilayers and their Thicknesses

Polymer layers and bilayers were prepared by spin coating (SCS 6800 Spin Coater) or casting. After the polymer was dissolved in Tetrachloroethane (TCE) or Chloroform, a volume of solution solution was loaded onto cover glass (12 mm or 24 mm) For making polymer bilayers, the casting or spin coating procedure was repeated on the dried polymer layer. After completely drying, thicknesses were determined by Profilometer (TABLES 1, 2, 3, 4, and 5).

In Table 1, polymer monolayers were formed by spin coating.

TABLE 1

| | Loading Vol. (μL)/ | Speed | Thickness |
|---|---|---|---|
| Substrate | 24 mm cover glass | (rpm) | (μm) |
| PCL-PEG (4:1) | 150 | 4000 | <3 |
| PCL-PEG (4:1) | 250 | 4000 | 3 |
| PCL-PEG (4:1) | 250 | 2000 | 5 |
| PCL-PEG (9:1) | 150 | 4000 | <3 |
| PCL-PEG (9:1) | 250 | 4000 | 3 |
| PCL-PEG (9:1) | 250 | 2000 | 5 |

In Table 2, PCL polymer monolayers were prepared by spin coating.

TABLE 2

| Conc. | Loading Vol. (μL)/ | Speed | Thickness |
|---|---|---|---|
| (mg/mL in TCE) | 24 mm cover glass | (rpm) | (μm) |
| 100 | 300 | 1500 | 3.1 |
| 100 | 200 | 1500 | 2.6 |
| 100 | 200 | 3000 | 2.1 |
| 100 | 200 | 800 | 6.5 |
| 200 | 200 | 1500 | 13.5 |
| 150 | 200 | 1500 | 7.4 |
| 200 | 200 | 1200 | 15.3 |
| 150 | 200 | 1200 | 9.8 |
| 100 | 200 | 1200 | 4.6 |
| 200 | 400 | 1200 | 24.0 |

In Table 3, PLGA polymer monolayers were prepared by spin coating.

TABLE 3

| Conc. | Loading Vol. (μL)/ | Speed | Thickness |
|---|---|---|---|
| (mg/mL in TCE) | 24 mm cover glass | (rpm) | (μm) |
| 100 | 200 | 1500 | 1.0 |
| 100 | 300 | 1500 | 0.8 |
| 200 | 200 | 1500 | 6.5 |
| 200 | 200 | 3000 | 2.3 |

In Table 4, PLGA/PCL polymer bilayers were prepared by spin coating.

TABLE 4

| | Loading Vol. (μL, | Speed | |
|---|---|---|---|
| Conc (PLGA/PCL) | PLGA/PCL)/24 | (rpm) | Thickness |
| (mg/mL in TCE) | mm cover glass | PLGA/PCL | (μm) |
| 100/100 | 300/200 | 1500/1500 | 2.1 |
| 100/100 | 300/200 | 3000/1500 | 1.7 |
| 100/100 | 300/200 | 1500/3000 | 1.7 |
| 100/150 | 300/200 | 1500/1500 | 4.5 |

In Table 5, polymer monolayers and bilayers were prepared by casting.

TABLE 5

| | Loading Vol. (μL)/ | Thickness |
|---|---|---|
| Substrate | 12 mm cover glass | (μm) |
| PLGA | 60 | 3.7 |
| PCL-PLGA Blend (1:1) | 60 | 3.6 |
| PCL-PEG Blend (1:1) | 70 | 18 |
| PCL | 70 | 8 |

### Example 6

### Permeability of Polymer Layers

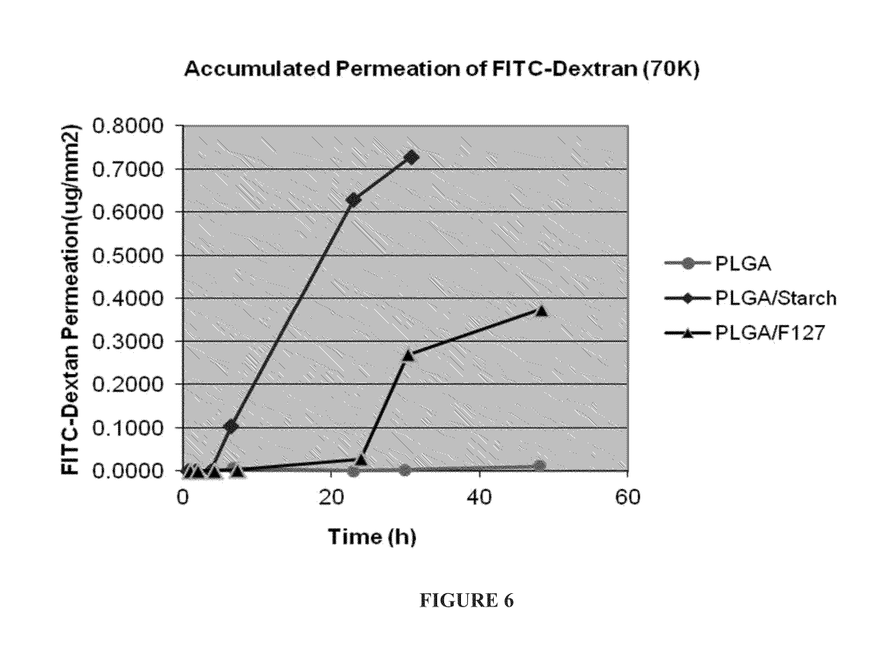

The in vitro permeability of polymer layers was evaluated by monitoring the concentration of FITC-Dextran (70K, Mw=70,000, Aldrich) diffused through the polymers in Blind-Well chamber (NeuroProbe, Inc.). 200 μL of a FITC-Dextran solution (1% in 0.5 mM PBS pH 7.2) was filled into the lower chamber, while the upper chamber was filled with 200 μL of the blank PBS. A scaffold was placed between two chambers. The concentration of dextran diffused into the upper chamber was measured by uv-spectroscopy, and monitored over time. The absorbance of these aliquots was analyzed by Plate Reader (Flex Station 3, Molecular Devices). PLGA layers (85:15, Mw: 50-75K, ˜10 μm) did not show any noticeable diffusion of dextran after 2 days. To improve the permeability of the PLGA layers, two blended PLGA layers, PLGA-starch (6:4) and PLGA-F127 (17:3) were prepared. PLGA-starch and PLGA-F127 blended layers showed a significant increase in diffusion of dextran after 7 h and 24 h respectively (FIG. 6). Additionally, both blended polymer layers retained their mechanical strength over the duration of the test. This result demonstrated that blended layers can give permeation, and allow to the transport of nutrients through the polymer layer.

### Example 7

### Subretinal Implantation of Polymer Scaffolds

All experiments were performed in compliance with the ARVO statement of the use of Animals in Ophthalmic and Vision Research under a protocol approved by the Animal Care and Use Committee (IACUC) at the University of Southern California. Dystrophic pigmented RCS rats (n=17) were obtained from in-house breeding and were transplanted at postnatal days (P) 27-29. Immunosuppression was obtained using topical prednisolone. Surgical procedure included a superior temporal peritomy, which was performed followed by fixation of the superior and temporal rectus muscles. A 0.9-1 2 mm sclerotomy was performed and a retinal detachment was induced injecting 10 μl of balanced salt solution into the subretinal space using a 32-gauge blunt needle. Before subretinal implantation the polymer layer was cut with surgical scissors from the edge for approximately a length of 1.0 mm. Using forceps the implant was placed in the subretinal space Immediately after the procedure, the correct placement of the implant was evaluated by Optical Coherence Tomography (OCT, Spectralis, Heidelberg, Germany) B-Scans.

Implanted animals were sacrificed at different time points between 5 to 18 days. After euthanization, the eyes of the rats were removed, fixed and H&E stained. Polymer layer thickness was assessed with the APERIO system. Slides were also processed for immunostaining.

### Results

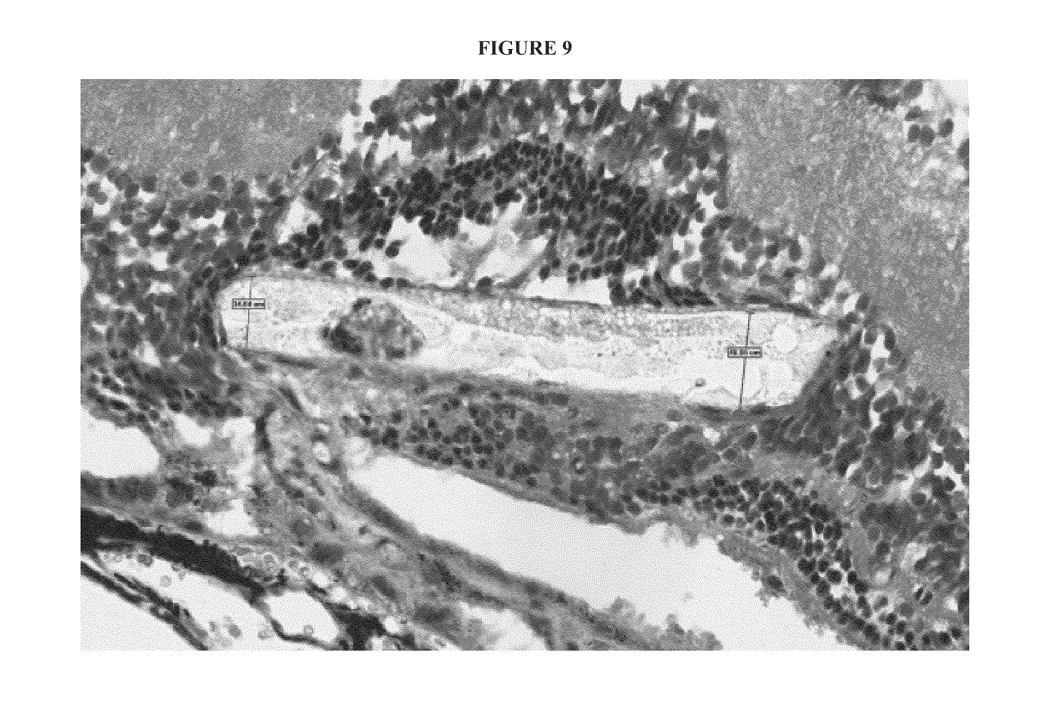

The thickness of the PLGA/PCL bilayer (PLGA top; PCL bottom) increased about 75% after 1 month (from about 25 micron to 40 micron) (FIG. 9). The PLGA and the PCL demonstrated pore formation and different degradation thicknesses depending on the layer.

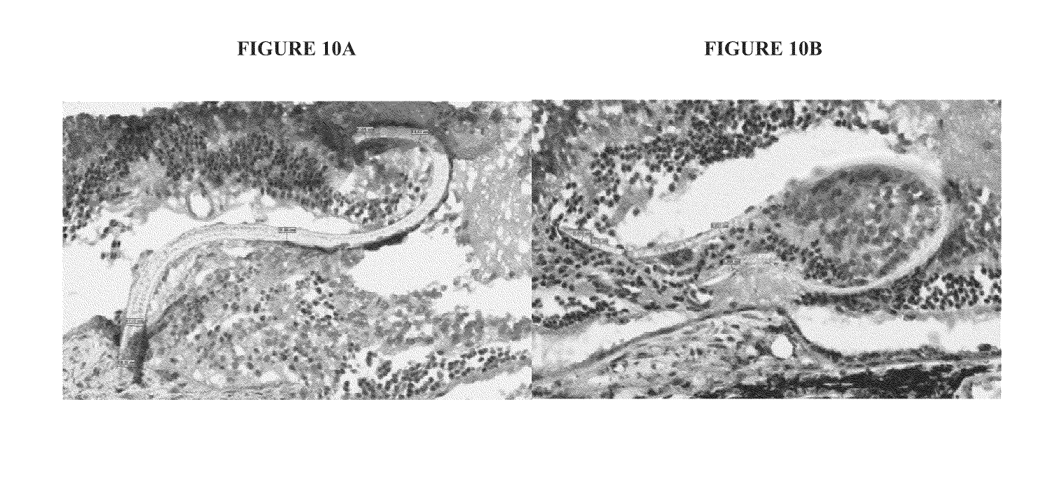

PLGA and PLA monolayers showed intra-retinal curling at 7 weeks and 6 weeks, respectively, post implantation (FIG. 10). Monolayer curling is likely the result of rapid degradation.

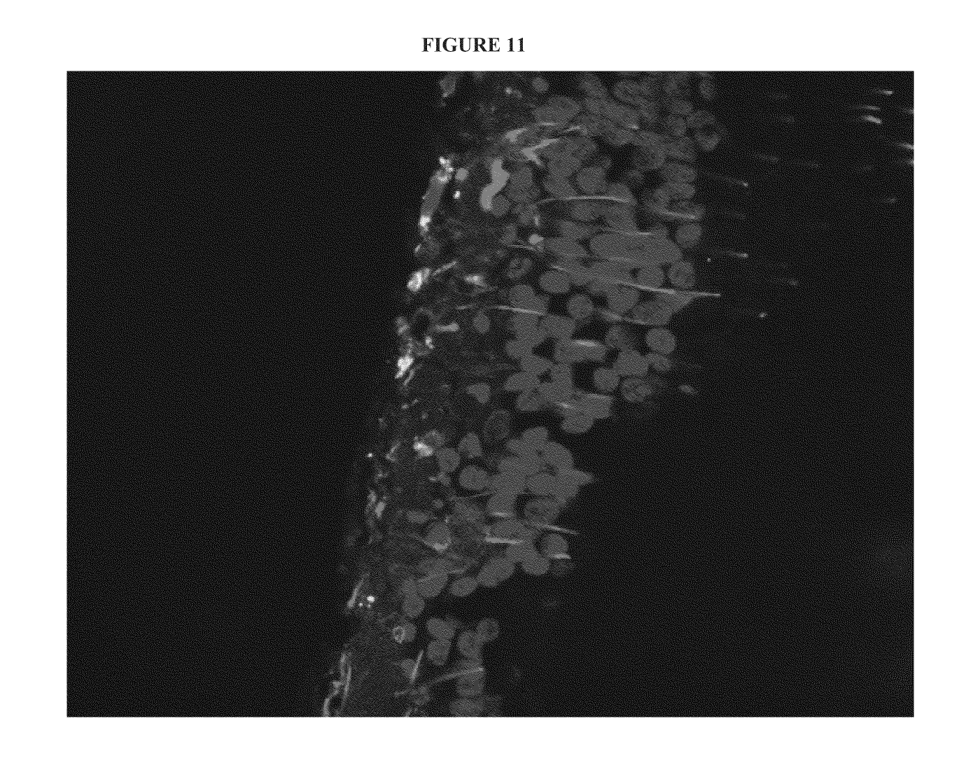

Immune reaction to the PLGA/PCL bilayer was investigated at 5 weeks after implantation by immunohistochemistry (FIG. 11). Some macrophages (green) and mild infiltration of the Mueller cells (red sparks) were observed. The outer nuclear layer (blue color) was shown to be unaffected.

The disclosures of each patent, patent application, and publication cited or described in this document are hereby incorporated herein by reference, in its entirety.

1. A scaffold for growing RPE cells, comprising:
two or more biodegradable polymers.

2. The scaffold of claim 1, wherein said two or more polymers comprise a blend.

3. The scaffold of claim 1, wherein said two or more polymers comprise two or more layers.

4-8. (canceled)

9. The scaffold of claim 1, further comprising an RGD peptide.

10-13. (canceled)

14. The scaffold of claim 9, wherein said RPE cells attach to said RDG peptide.

15-21. (canceled)

22. An RGD peptide linked polymer scaffold for supporting the growth of RPE cells, comprising: an RGD peptide bonded to a linker, the linker being bonded to a polymer scaffold.

23-39. (canceled)

40. The RGD peptide linked polymer scaffold of claim 22, wherein said polymer scaffold comprises a single layer of polymer.

41. The RGD peptide linked polymer scaffold of claim 22, wherein said polymer scaffold comprises multiple layers of polymers.

42-43. (canceled)

44. The RGD peptide linked polymer scaffold of claim 41, wherein said multiple layer scaffold is coated with different polymers, copolymers or blends of polymers such that each layer possesses a different biodegradable rate.

45-49. (canceled)

50. A method of creating a scaffold for growing RPE cells, comprising:
linking an RGD peptide to a polymer scaffold, wherein said polymer is modified and crosslinked.

51-72. (canceled)

73. The method of claim 50, wherein said polymer is covalently modified with a diamine.

74. (canceled)

75. The method of claim 50, wherein said polymer is crosslinked at a temperature range of about 10° C. to about 50° C.

76-78. (canceled)

79. The method of claim 50, wherein said RGD peptide is linked to said polymer at a temperature range of about 10° C. to about 50° C.

80-83. (canceled)

84. A method of culturing RPE cells, comprising: growing said RPE cells on an RGD peptide linked polymer scaffold such that said cells attach to said scaffold and remain differentiated.

85-93. (canceled)

94. The method of claim 84, wherein said attached RPE cells continue to proliferate and form a monolayer.

95-100. (canceled)

101. The method of claim 84, wherein said RPE cells comprise embryonic stem cell derived RPE cells, induced pluripotent stem cell derived RPE cells, adult stem cell derived RPE cells, maturated RPE cells, or any combination thereof.

102-103. (canceled)

104. The method of claim 84, wherein said RGD peptide comprises a cyclic RGD peptide, non-cyclic RGD peptide, a protein containing an RGD sequence, or any combination thereof.

105-106. (canceled)

107. A method of treating vision loss in a subject, comprising:
administering a RPE cell attached RGD peptide linked polymer scaffold to said subject in need of treatment.

108-135. (canceled)

136. The method of claim 107, wherein said RPE cell attached polymer scaffold is administered to the eye of said subject.

137-144. (canceled)

145. The method of claim 107, wherein said administered RPE cell attached polymer scaffold remains permeable to nutrients and oxygen.

146. The method of claim 107, wherein said polymer scaffold degrades following administration.

147-150. (canceled)

151. The method of claim 146, wherein following degradation of said polymer scaffold said RPE cells are incorporated into the eye of the subject.

152-154. (canceled)

155. The method of claim 107, wherein said polymer scaffold is coated with an agent to prevent an adverse reaction in the subject undergoing treatment.

156-158. (canceled)

159. A kit, comprising:
a support surface, one or more polymers, a linker, a crosslinking agent, RGD peptide, and RPE cells.

160-178. (canceled)
